(19) United States
(12) Patent Application Publication
(10) Pub. No.: US 2026/0209262 A1
(43) Pub. Date: Jul. 23, 2026

(54) MEMBRANE FILTRATION-ASSISTED SOLUTION PHASE OLIGONUCLEOTIDE SYNTHESIS

(71) Applicant: EXACTMER LIMITED, Harrogate Yorkshire (GB)

(72) Inventors: Andrew Guy Livingston, Harrogate (GB); Piers Gaffney, Harrogate (GB)

(21) Appl. No.: 19/138,953

(22) PCT Filed: Dec. 14, 2023
(86) PCT No.: PCT/GB2023/053244

§ 371 Date: Jun. 13, 2025

(30) Foreign Application Priority Data
Dec. 16, 2022 (GB) 2219099.5

(51) Int. Cl. C07H 21/00; B01D 71/62; C07H 1/06

(52) U.S. Cl. CPC C07H 21/00; B01D 71/62; C07H 1/06

(57) ABSTRACT

The present invention relates to a membrane filtration-assisted process for the preparation of oligonucleotides by solution-phase synthesis. In particular, the present invention relates to a solution-phase process for preparing oligonucleotides in which membrane filtration (e.g., diafiltration) is used to purify and/or isolate the oligonucleotide during its step-wise growth.

### INTRODUCTION

The present invention relates to a membrane filtration-assisted process for the preparation of oligonucleotides by solution-phase synthesis. In particular, the present invention relates to a solution-phase process for preparing oligonucleotides in which membrane filtration (e.g., diafiltration) is used to purify and/or isolate the oligonucleotide during its stepwise growth.

### BACKGROUND OF THE INVENTION

Oligonucleotide-based drugs have previously been advanced as a new generation of therapeutics functioning at the protein expression level and have recently been validated as a new pharmaceutical modality for treating a wide range of serious or life-threatening indications. Oligonucleotides are formed from a backbone of ribose phosphate monomers, with each monomer having a variable nucleobase side chain; the building block unit of ribose phosphate bound to a nucleobase constitutes a nucleotide. The precise sequence of nucleotides defines the oligonucleotide's biological function.

For many years oligonucleotides have been prepared using solid-phase oligonucleotide synthesis (SPOS) wherein a growing oligonucleotide is tethered to an insoluble solid support and grown by flowing nucleotide building blocks over the insoluble solid support. Although this method has been the industry standard, there are a number of drawbacks associated with SPOS. In particular, an excess of nucleotide building block is commonly required in order to drive the reaction to completion, which raises the cost of SPOS considerably. Moreover, scaling up of SPOS reactions is limited to producing only ~15 kg of oligonucleotide per batch. This is particularly undesirable for use as a pharmaceutical modality, wherein tonnes of oligonucleotide per annum would be required for a major medical indication (e.g., cardiovascular disease).

One alternative strategy to SPOS which aims to address these scale-up and economic challenges has been liquid phase oligonucleotide synthesis (LPOS). Indeed, liquid phase reactions and liquid phase material handling are established technologies that can be performed at the multi-tonne scale, making LPOS a strong candidate for oligonucleotide preparation at scale. A typical approach to LPOS is to carry out sequential coupling reactions, adding monomers or multi-monomer oligomers (fragments) to a growing oligonucleotide in solution in a stepwise fashion, and then to use a suitable separation technology, such as membrane filtration, to separate unreacted monomers or fragments from the growing oligonucleotide.¹⁻¹⁰

After the step of coupling a monomer or fragment onto a growing oligonucleotide, membrane filtration can be used to separate the unreacted monomers/fragments and any reaction debris from the growing oligonucleotide. The use of membrane filtration is therefore well-suited for LPOS, wherein a thorough purification step is preferred after each coupling step. Membrane filtration is particularly useful when the process for preparing oligonucleotides is to be conducted in a single organic solvent system (e.g., acetonitrile).

Although this technology has addressed many of the challenges faced by SPOS, some issues with using membrane filtration separation in LPOS remain. Most notably, when preparing lengthy oligonucleotides (i.e., ≥8 nucleotides in length) formed from a plurality coupling reactions, purification by membrane filtration becomes increasingly arduous as the oligonucleotide grows in length. In such circumstances, the viscosity of the solution may increase to such an extent that membrane flux drops to an unacceptably low level, and the rejection of the growing oligonucleotide may drop due to concentration polarisation, making it difficult and inefficient, if not impossible to isolate the growing oligonucleotide, thereby compromising any further coupling reactions. Moreover, the rise in viscosity significantly increases the likelihood of membrane fouling, thereby necessitating complete replacement of the membrane, or even selection of a new, looser membrane. To date, attempts to address this problem have focused on using excessively large volumes of solvent to permit additional coupling steps. However, even when new solvent is added, the membrane may still continue to foul during later filtration steps. There is therefore a need to improve membrane filtration-assisted processes for preparing oligonucleotides in solution, particularly where the target oligonucleotide is of increased length. The present invention was devised with the foregoing in mind.

### SUMMARY OF THE INVENTION

According to a first aspect of the present invention, there is provided a solution-phase process for the preparation of an oligonucleotide, the process comprising the step of:
growing the oligonucleotide by performing one or more sequential coupling reactions, each sequential coupling reaction increasing the length of the growing oligonucleotide by at least one nucleotide,
   wherein the growing oligonucleotide comprises at least one protected uracil nucleobase and/or at least one protected thymine nucleobase, and
   wherein the step of growing the oligonucleotide comprises one or more membrane filtration steps to isolate the growing oligonucleotide.

According to a second aspect of the present invention, there is provided an oligonucleotide obtained, directly obtained or obtainable by the process of the first aspect.

### DETAILED DESCRIPTION OF THE INVENTION

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

As described hereinbefore, in a first aspect, the present invention provides a solution-phase process for the preparation of an oligonucleotide, the process comprising the step of:
growing the oligonucleotide by performing one or more sequential coupling reactions, each sequential coupling reaction increasing the length of the growing oligonucleotide by at least one nucleotide,
   wherein the growing oligonucleotide comprises at least one protected uracil nucleobase and/or at least one protected thymine nucleobase, and
   wherein the step of growing the oligonucleotide comprises one or more membrane filtration steps to isolate the growing oligonucleotide.

Through rigorous investigations, the inventors have devised a vastly improved membrane filtration-assisted process for the preparation of oligonucleotides by solution-phase synthesis. In particular, the inventors have found that the presence of at least one protected uracil nucleobase and/or at least one protected thymine nucleobase on the growing oligonucleotide can significantly reduce the viscosity of the oligonucleotide solution. The surprising reduction in viscosity imparted by the at least one protected uracil nucleobase and/or at least one protected thymine nucleobase mitigates the drawbacks typically associated with conventional membrane filtration-assisted LPOS techniques, including membrane fouling and poor membrane flux. Indeed, the inventors have surprisingly found that the solution-phase process of the present invention allows for the straightforward preparation of oligonucleotides of increased length (e.g., ≥8 nucleotides in length) by a plurality of sequential coupling reactions in certain industry-favoured solvents.

Oligonucleotides will be familiar to those skilled in the art and will be understood to comprise a plurality of nucleotides linked (i.e., coupled) to one another to form a nucleotide sequence. As described herein, oligonucleotides are prepared by the stepwise addition of monomeric, dimeric and/or oligomeric nucleotidic building blocks to a growing oligonucleotide, with each addition being referred to as a coupling reaction. In the context of the present invention, the term “oligonucleotide” refers to the sequence of nucleotides after the final coupling reaction (sometimes termed the full-length product in industry), whereas the term “growing oligonucleotide” refers to the sequence of nucleotides up to (and during) the final coupling reaction. In its simplest sense, the “growing oligonucleotide” may be a single nucleotide.

The growing oligonucleotide may be a single growing oligonucleotide or a plurality (e.g., 2-12) of growing oligonucleotides Suitably, the growing oligonucleotide is 2-10 growing oligonucleotides. More suitably, the growing oligonucleotide is 2-8 growing oligonucleotides. Most suitably, the growing oligonucleotide is 3-4 growing oligonucleotides. Accordingly, the step of growing the oligonucleotide may comprise growing a plurality (e.g., 2-12) of oligonucleotides. Suitably, the step of growing the oligonucleotide comprises growing 2-10 oligonucleotides. More suitably, the step of growing the oligonucleotide comprises growing 2-8 oligonucleotides. Most suitably, the step of growing the oligonucleotide comprises growing 3-4 oligonucleotides. It will be understood that in embodiments wherein the growing oligonucleotide is a plurality of growing oligonucleotides, each growing oligonucleotide has an identical sequence of nucleotides.

Each oligonucleotide, once prepared (i.e., grown to full-length product), may have a molecular weight of ≥1000 Da. Suitably, each oligonucleotide has a molecular weight of ≥2000 Da. More suitably, each oligonucleotide has a molecular weight of ≥3000 Da. Even more suitably, each oligonucleotide has a molecular weight of ≥5000 Da.

The growing oligonucleotide comprises at least one protected uracil nucleobase and/or at least one protected thymine nucleobase. The term “protected” in the context of the present invention will be understood to mean that the nucleobase comprises a protecting group (i.e., at least one protecting group). Accordingly, the growing oligonucleotide comprises: (i) at least one protected uracil nucleobase, wherein the protected uracil nucleobase comprises a protecting group; and/or (ii) at least one protected thymine nucleobase, wherein the protected thymine nucleobase comprises a protecting group. Protecting groups will be familiar to those skilled in the art and will be understood to mean an organic moiety attached to a functional group in order to block the reactivity of said functional group.

The location of the protecting group is dependent on the nature of the protected nucleobase. As will be familiar to those skilled in the art, uracil nucleobases adopt the following configurations in oligonucleotides:

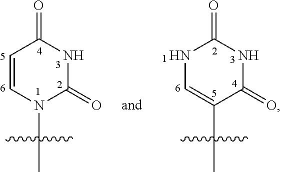

wherein [symbol] denotes the point of attachment to the growing oligonucleotide.
Thymine nucleobases adopt the following configuration in oligonucleotides:

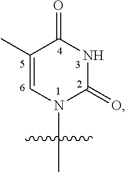

wherein [symbol] denotes the point of attachment to the growing oligonucleotide.

Suitably, in all protected uracil and thymine nucleobases defined herein, [symbol] denotes the point of attachment to a five-carbon sugar (e.g., ribose or deoxyribose) in the growing oligonucleotide. Some examples of protected uracil and protected thymine attached to a five carbon sugar are shown below:

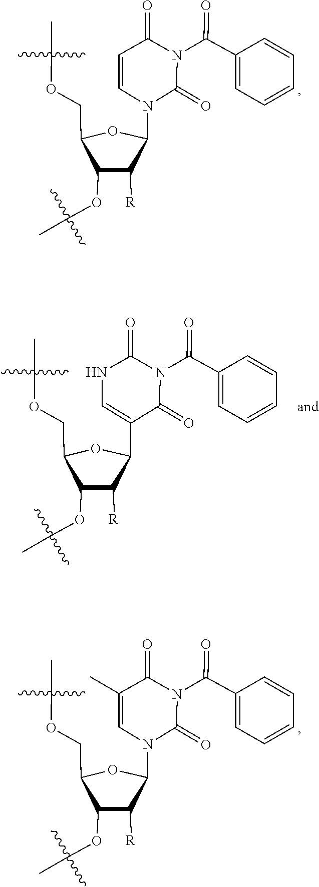

wherein [symbol] denotes the point of attachment to the remainder of the growing oligonucleotide, and R is H, OH, OMe or F.

As depicted hereinbefore, protected uracil nucleobases and protected thymine nucleobases can be attached to the growing oligonucleotide at the N1 position (e.g., uridine and thymidine). In such embodiments, the protecting group may be located at the N3 position or the O4 position (i.e., the oxygen atom bound to C4). Accordingly, when the at least one protected uracil nucleobase comprises a protecting group and is attached to the growing oligonucleotide at the N1 position, the protecting group is located at the N3 position or the 04 position. Similarly, when the at least one protected thymine nucleobase comprises a protecting group and is attached to the growing oligonucleotide at the N1 position, the protecting group is located at the N3 position or the 04 position. Suitably, in both of the aforementioned embodiments, the protecting group is located at the N3 position.

The nature of the protecting group will depend on whether the protecting group is located at the N3 position or the 04 position on the uracil nucleobase and/or the thymine nucleobase. For example, certain protecting groups may initially be located at the 04 position (the kinetic product), but may subsequently migrate to the N3 position (the thermodynamic product).

Alternatively, protected uracil nucleobases can be attached to the growing oligonucleotide at the C5 position (e.g., pseudouridine). In such embodiments, the protecting group may be located at the N3 position or the 02 position (i.e., the oxygen atom bound to C2). Accordingly, when the at least one protected uracil nucleobase comprises a protecting group and is attached to the growing oligonucleotide at the C5 position, the protecting group is located at the N3 position or the O2 position. In this embodiment, an additional protecting group may also be located at the N1 position.

In embodiments wherein the protected uracil nucleobase is attached to the growing oligonucleotide at the C5 position, the protecting group may be located at the N1 position or the O2 position (i.e., the oxygen atom bound to C2). Accordingly, when the at least one protected uracil nucleobase comprises a protecting group and is attached to the growing oligonucleotide at the C5 position, the protecting group is located at the N1 position or the 02 position. In this embodiment, an additional protecting group may also be located at the N3 position.

As discussed hereinbefore, the inventors have found that the protecting groups of the protected uracil nucleobase and/or the protected thymine nucleobase surprisingly play an important role in reducing the viscosity of the solution during membrane filtration. In particular, by reducing the viscosity of the solution, membrane fouling and a reduction in membrane flux can be mitigated. This avoids the need for replacing the membrane or selecting a looser membrane during purification and isolation, which can be costly and time consuming. Furthermore, the use of excessively large volumes of solvent to reduce building block concentration is avoided.

Each protecting group may independently be an acid-labile protecting group, a base labile protecting group, an ammonia labile protecting group, an oximate labile protecting group, an oxidatively labile protecting group, a hydrogenolytically labile protecting group or a transition metal catalysed cleavage protecting group. Suitably, each protecting group is independently selected from the group consisting of 2,4,6-trimethylphenyl, 2-nitrophenyl, 2,4-dimethylphenyl, toluyl, 2-(4-nitrophenyl)ethyl, 2-(4-cyanophenyl)ethyl, allyl, benzoyl (Bz), 2,4-dimethylbenzoyl, tert-Bu benzoyl (tert-BuBz), acetyl (Ac), anisoyl (An), 4-chlorobenzoyl, diphenylcarbamoyl, butylthiocarbonyl, 2-nitrophenylsulfenyl, 2,4-dinitrophenylsulfenyl, 2-nitro-4-toluylsulfenyl, and triphenylmethylsulfenyl. The protecting groups may be located on the protected uracil nucleobase and/or the thymine nucleobase in the positions described hereinbefore. Suitably, each protecting group is independently selected from the group consisting of benzoyl (Bz), 2,4-dimethylbenzoyl, tert-Bu benzoyl (tert-BuBz) and anisoyl (An).

It will be understood that in embodiments wherein the growing oligonucleotide comprises more than one protected uracil nucleobase, each uracil nucleobase may be independently attached to the growing oligonucleotide at different positions (i.e., one protected uracil nucleobase at the N1 position and another protected uracil nucleobase at the C5 position).

In the step of growing the oligonucleotide, the oligonucleotide is grown by performing one or more sequential coupling reactions, each sequential coupling reaction increasing the length of the growing oligonucleotide by at least one nucleotide. The coupling reactions may be monomeric, dimeric or oligomeric in nature. For example, a coupling reaction may involve adding a monomeric building block to each growing oligonucleotide (i.e., the length of the growing oligonucleotide is increased by a single nucleotide in a single coupling reaction). Alternatively, a coupling reaction may involve adding a dimeric building block (i.e., two pre-coupled nucleotides) to each growing oligonucleotide (i.e., the length of the growing oligonucleotide is increased by two nucleotides in a single coupling reaction). Alternatively, a coupling reaction may involve adding an oligomeric building block (i.e., three or more pre-coupled nucleotides) to each growing oligonucleotide (i.e., the length of the growing oligonucleotide is increased by three or more nucleotides in a single coupling reaction).

Each sequential coupling reaction will be independent of each other. Therefore, it may be that the step of growing the oligonucleotide by performing one or more sequential coupling reactions comprises a coupling reaction which increases the length of the growing oligonucleotide by a single nucleotide and a coupling reaction which increases the length of the growing oligonucleotide by two nucleotides. It may be that the step of growing the oligonucleotide by performing one or more sequential coupling reactions comprises a coupling reaction which increases the length of the growing oligonucleotide by a single nucleotide and a coupling reaction which increases the length of the growing oligonucleotide by three or more nucleotides. It may also be that the step of growing the oligonucleotide by performing one or more sequential coupling reactions comprises a coupling reaction which increases the length of the growing oligonucleotide by two nucleotides and a coupling reaction which increases the length of the growing oligonucleotide by three or more nucleotides.

In its simplest sense, the step of growing the oligonucleotide may comprise only a single coupling reaction, for example between an initial monomeric unit (i.e., the growing oligonucleotide) and a further monomeric, dimeric or oligomeric building block (i.e., a single nucleotide, two pre-coupled nucleotides or three or more nucleotides, respectively). The step of growing the oligonucleotide may comprise performing two or more sequential coupling reactions. Suitably, the step of growing the oligonucleotide comprises performing three or more sequential coupling reactions. More suitably, the step of growing the oligonucleotide comprises performing four or more sequential coupling reactions. Even more suitably, the step of growing the oligonucleotide comprises performing six or more sequential coupling reactions. Yet more suitably, the step of growing the oligonucleotide comprises performing ten or more sequential coupling reactions. Most suitably, the step of growing the oligonucleotide comprises performing fifteen or more sequential coupling reactions.

The step of growing the oligonucleotide may comprise a plurality of sequential coupling reactions, wherein each sequential coupling reaction increases the length of the growing oligonucleotide by one nucleotide. Suitably, the plurality of sequential coupling reactions is two or more sequential coupling reactions. More suitably, the plurality of sequential coupling reactions is three or more sequential coupling reactions. Yet more suitably, the plurality of sequential coupling reactions is four or more sequential coupling reactions. Even more suitably, the plurality of sequential coupling reactions is six or more sequential coupling reactions. Yet still even more suitably, the plurality of sequential coupling reactions is ten or more sequential coupling reactions. Most suitably, the plurality of sequential coupling reactions is fifteen or more sequential coupling reactions.

Each coupling reaction typically involves reacting a free (unprotected) terminal of a growing oligonucleotide with a reactive terminal of a monomeric, dimeric or oligomeric building block to be coupled, and subsequently deprotecting the terminal of the newly coupled monomeric, dimeric or oligomeric building block to generate a new free (unprotected) terminal (in preparation for performing a subsequent coupling reaction). The skilled person will be familiar with protecting groups used to prevent uncontrolled polymer chain extension in the solution-phase synthesis of oligonucleotides, as well as the manner in which they can be removed.

The reactive site of a monomeric, dimeric or oligomeric building block to be coupled (i.e., a single nucleotide, two pre-coupled nucleotides or three or more nucleotides, respectively) to the distal terminus of a growing oligonucleotide chain may consist of a phosphoramidite, a phosphate monoester, a phosphate diester, an H-phosphonate, a cyclic thiophosphate or a cyclic dithiophosphate triester moiety, or any other phosphorus containing precursor to the internucleotide linkage, or any other species leading to an analogue of the internucleotide linkage well known to the skilled person familiar with oligonucleotide chemical synthesis.¹²

The step of growing the oligonucleotide may be conducted in at least one organic solvent. Suitably, the step of growing the oligonucleotide is conducted in acetonitrile, optionally mixed with another organic solvent. More suitably, the step of growing the oligonucleotide is conducted in acetonitrile mixed with sulfolane. In such embodiments, the ratio of acetonitrile to sulfolane may be 4:1 v/v. Alternatively, in some embodiments, the step of growing the oligonucleotide is conducted in acetonitrile (i.e., neat acetonitrile). Acetonitrile is the solvent favoured by industry for coupling nucleotides to prepare oligonucleotides. Most suitably, the step of growing the oligonucleotide is conducted in neat acetonitrile or acetonitrile mixed with sulfolane (e.g., 4:1 v/v).

In some embodiments, the step of growing the oligonucleotide comprises two or more sequential coupling reactions and is conducted in at least one organic solvent. In some embodiments, the step of growing the oligonucleotide comprises four or more sequential coupling reactions and is conducted in acetonitrile optionally mixed with another organic solvent. In some embodiments, the step of growing the oligonucleotide comprises six or more sequential coupling reactions and is conducted in neat acetonitrile or acetonitrile mixed with sulfolane (e.g., 4:1 v/v).

The step of growing the oligonucleotide comprises one or more membrane filtration steps to isolate the growing oligonucleotide. Suitably, the membrane filtration is membrane diafiltration. Most suitably, the membrane filtration is organic solvent nanofiltration (OSN) or ultrafiltration (UF). Membrane filtration may be performed to separate the growing oligonucleotide from a reaction by-product formed as part of a coupling reaction (e.g., a protecting group cleaved from the terminal of the growing oligonucleotide) or from an excess reagent used as part of a coupling reaction (e.g., an excess of a monomeric, dimeric or oligomeric building block to be coupled). The growing oligonucleotide, excess reagent and reaction by-product remain in solution during the step of growing the oligonucleotide.

Membrane filtration may be performed once or twice for a given coupling reaction. A first filtration may involve separating the growing oligonucleotide from a reaction by-product formed as part of a coupling reaction (e.g., a protecting group cleaved from the terminal of the growing oligonucleotide). A second filtration may involve separating the growing oligonucleotide from an excess reagent used as part of a coupling reaction (e.g., an excess of a monomeric, dimeric or oligomeric building block to be coupled). Suitably, membrane filtration is performed twice per coupling reaction.

Membrane filtration need not be performed as part of every sequential coupling reaction conducted in the step of growing the oligonucleotide. For example, if the step of growing the oligonucleotide comprises 3 sequential coupling reactions, membrane filtration may be performed as part of only 1 or 2 of these reactions. In some embodiments, however, membrane filtration is performed as part of every sequential coupling reaction conducted in the step of growing the oligonucleotide.

The step of growing the oligonucleotide and the one or more membrane filtration steps to isolate the growing oligonucleotide may be conducted in the same solvent. Suitably, the step of growing the oligonucleotide and the one or more membrane filtration steps are conducted in at least one organic solvent. More suitably, the step of growing the oligonucleotide and the one or more membrane filtration steps are conducted in acetonitrile, optionally mixed with another organic solvent. Yet more suitably, the step of growing the oligonucleotide and the one or more membrane filtration steps are conducted in acetonitrile mixed with sulfolane. In such embodiments, the ratio of acetonitrile to sulfolane may be 4:1 v/v. Alternatively, in some embodiments, the step of growing the oligonucleotide and the one or more membrane filtration steps are conducted in neat acetonitrile. Most suitably, the step of growing the oligonucleotide and the one or more membrane filtration steps to isolate the growing oligonucleotide are conducted in neat acetonitrile or acetonitrile mixed with sulfolane (e.g., 4:1 v/v).

Suitable membranes for use in the one or more membrane filtration steps to isolate the growing oligonucleotide include polymeric membranes, ceramic membranes, and mixed polymeric/inorganic membranes. Membrane rejection Rᵢ is a common term known by those skilled in the art and is defined as:

$${R}_{i} = ( 1 - \frac{{C}_{Pi}}{{C}_{Ri}} ) \times 100 \% \qquad eq . ( 1 )$$

where C_{P,i}=concentration of species i in the permeate, permeate being the liquid which has passed through the membrane, and C_{R,i}=concentration of species i in the retentate, retentate being the liquid which has not passed through the membrane. It will be appreciated that a membrane is suitable for the invention if

$${R}_{( growing oligonucleutide )} > {R}_{( at least one reaction by - product or reagent )}$$

Typically, during the one or more membrane filtration steps, the crude mixture comprising the growing oligonucleotide is pressurised against a size-selective solvent stable membrane. Here, the soluble synthesis support plays a role beyond being a passive solubility aid. During the process of diafiltration (separation of solutes by permeating the solution through a selective membrane) solutes that exhibit any rejection by the membrane accumulate on the retentate side at the interface between the bulk solution and the membrane. The soluble synthesis support is designed to have the highest possible, preferably 100%, rejection by the membrane. Excess reagents used in the coupling reaction(s) (e.g., nucleotides) typically have the next highest molecular weight, such that they typically have the next highest rejection. It is important to remove all excess reagent prior to beginning the next coupling reaction so as to prevent the free hydroxyl group of a retained nucleotide providing a site for unwanted growth of a truncated oligomeric contaminant.

To achieve the highest possible purity of the oligonucleotide (i.e., once fully grown), the highest possible coupling efficiency is desirable. Given that the rate of the bimolecular reaction of a hydroxy terminus of a growing oligonucleotide with a nucleotide building block is approximately proportional to the concentration of both species, the highest practical concentration of both the growing oligonucleotide and building block should be achieved to allow the process to achieve near quantitative conversion. Since a high building block concentration could be used to drive reactions nearer to 100% completion, a large excess of building block may seem advantageous. However, both for economical oligonucleotide synthesis (for example, nucleotide building blocks are very costly) and to minimise the amount of debris that must be removed by diafiltration, a low excess of building block is preferred and therefore highly desirable. Furthermore, a low excess of building block minimises the amount of diafiltration solvent required to achieve a specified purity of oligonucleotide; typically, 0.1% of the initial concentration of residual building block (=0.001×0.5 equivalents) is acceptable.

As a compromise between these two extremes, a 4 to 40 mM concentration of growing oligonucleotide is desirable for rapid coupling in acetonitrile/acetonitrile mixed with sulfolane with an economical excess of 1-2 (e.g., 1.5) equivalents of oligonucleotide (e.g., phosphoramidite) building block. LPOS phosphoramidite chain extension reactions can be initiated with common activators, such as ethylthiotetrazole (ETT) or dicyanoimidazole (DCI), and proceed for 5 to 20 minutes. The reaction may be quenched with a small excess of an alcohol or water, then oxidation (using agents such as camphorsulfonyl oxaziridine (CSO), cumenyl hydroperoxide, or tert-butyl hydroperoxide) or sulfur transfer (using agents such as phenylacetyl disulfide (PADS), or xanthane hydride (XH), or 3-phenyl 1,2,4-dithiazoline-5-one (POS)) undertaken, after which the crude mixture can be purified by OSN.

The membranes useful in the one or more membrane filtration steps may be formed from any polymeric or ceramic material which provides a separating layer capable of preferentially separating the growing oligonucleotide from at least one reaction by-product or reagent used in the step of growing the oligonucleotide. In other words, the membrane will exhibit a rejection for the growing oligonucleotide that is greater than the rejection for the reaction by-product or reagent. Suitably, the membrane is formed from or comprises a polymeric material suitable for fabricating microfiltration, ultrafiltration, nanofiltration or reverse osmosis membranes, including polyethylene, polypropylene, polytetrafluoroethylene (PTFE), polyvinylidene difluoride (PVDF), polysulfone, polyethersulfone, polyacrylonitrile, polyamide, polyester, polyimide, polyetherimide, cellulose acetate, polyaniline, polypyrrole, polybenzimidazole, polyetheretherketone (PEEK) and mixtures thereof. The membranes can be made by any technique known in the art, including sintering, stretching, track etching, template leaching, interfacial polymerisation or phase inversion. Membranes may be composite in nature (e.g., a thin film composite membrane) and/or be crosslinked or treated so as to improve their stability in the solvent used. PCT/GB2007/050218, PCT/GB2015/050179 and U.S. Pat. No. 10,913,033 describes membranes useful in the one or more membrane filtration steps. The membrane will be stable in the organic solvent system used in the step of growing the oligonucleotide and the one or more membrane filtration steps to isolate the growing oligonucleotide.

Suitably, the one or more membrane filtration steps is performed using a crosslinked polybenzimidazole membrane (e.g., an integrally skinned, asymmetric, crosslinked polybenzimidazole membrane) or a polyetheretherketone membrane.

In some embodiments, the membrane filtration is performed once or twice per coupling reaction and is performed using a crosslinked polybenzimidazole membrane or a polyetheretherketone membrane. In some embodiments, the membrane filtration is performed twice per coupling reaction and is performed using a crosslinked polybenzimidazole membrane.

During the step of growing the oligonucleotide, the growing oligonucleotide may be attached at one end to a soluble synthesis support. The nature of the attachment between the growing oligonucleotide and the soluble synthesis support may be direct or indirect (e.g., via a linker). A variety of soluble synthesis supports capable of solubilising the oligonucleotide during growth may be used. The soluble synthesis support may comprise a central hub and one or more solubility-enhancing polymers, each attached to the central hub. The nature of the attachment between each solubility-enhancing polymer and the central hub may be direct or indirect (e.g., via a linker). Each growing oligonucleotide may be attached directly or indirectly (e.g., via a linker) at one of its ends to the central hub, to a solubility-enhancing polymer, or to any linker that may be linking the central hub to a solubility-enhancing polymer. Suitably, each growing oligonucleotide is attached directly or indirectly (e.g., via a linker) at one of its ends to a solubility-enhancing polymer. The growing oligonucleotide attached to the soluble synthesis support may be referred to herein as the supported growing oligonucleotide.

In particular embodiments, the one or more solubility-enhancing polymers are each attached to the central hub and each growing oligonucleotide is attached at one of its ends to a solubility-enhancing polymer. Suitably, within each molecule of soluble synthesis support, the number of solubility-enhancing polymers is equal to the number of growing oligonucleotides.

The one or more solubility-enhancing polymers may be a single solubility-enhancing polymer or a plurality (e.g., 2-12) of solubility-enhancing polymers (i.e., each molecule of soluble synthesis support may comprise a plurality of solubility-enhancing polymers). Suitably, the one or more solubility-enhancing polymers is 2-10 solubility-enhancing polymers. More suitably, the one or more solubility-enhancing polymers is 2-8 solubility-enhancing polymers. Most suitably, the one or more solubility-enhancing polymers is 3-4 solubility-enhancing polymers.

In some embodiments, the growing oligonucleotide is attached at one end to a soluble synthesis support, wherein the soluble synthesis support comprises a central hub and a plurality (e.g., 2-12) of solubility-enhancing polymers, each attached to the central hub. In some embodiments, the growing oligonucleotide is attached at one end to a soluble synthesis support, wherein the soluble synthesis support comprises a central hub and 2-8 of solubility-enhancing polymers, each attached to the central hub. In some embodiments, the growing oligonucleotide is attached at one end to a soluble synthesis support, wherein the soluble synthesis support comprises a central hub and 3-4 solubility-enhancing polymers, each attached to the central hub.

In some embodiments, the growing oligonucleotide is attached at one end to a soluble synthesis support, wherein the soluble synthesis support comprises a central hub and a plurality (e.g., 2-12) of solubility-enhancing polymers, each attached to the central hub, and wherein the number of solubility-enhancing polymers is equal to the number of growing oligonucleotides. In some embodiments, the growing oligonucleotide is attached at one end to a soluble synthesis support, wherein the soluble synthesis support comprises a central hub and 2-8 of solubility-enhancing polymers, each attached to the central hub, and wherein the number of solubility-enhancing polymers is equal to the number of growing oligonucleotides. In some embodiments, the growing oligonucleotide is attached at one end to a soluble synthesis support, wherein the soluble synthesis support comprises a central hub and 3-4 solubility-enhancing polymers, each attached to the central hub, and wherein the number of solubility-enhancing polymers is equal to the number of growing oligonucleotides.

Where each growing oligonucleotide is attached to a solubility-enhancing polymer via a linker, a range of chemistries is available to construct this linkage. For example, if the solubility-enhancing polymer terminates with hydroxyl functionality, this can be esterified with a nucleoside succinate. If the solubility-enhancing polymer terminates with an amino functionality this can be condensed directly with a nucleoside succinate to form a succinate ester-amide. Other linkages may have greater stability; for instance, a PEG-amine can be reacted with Fmoc-sarcosine, or Boc-sarcosine, then deprotected to leave a secondary N-methyl poly(ethylene glycol) chain terminus. Scheme 1 below illustrates various suitable linkers:

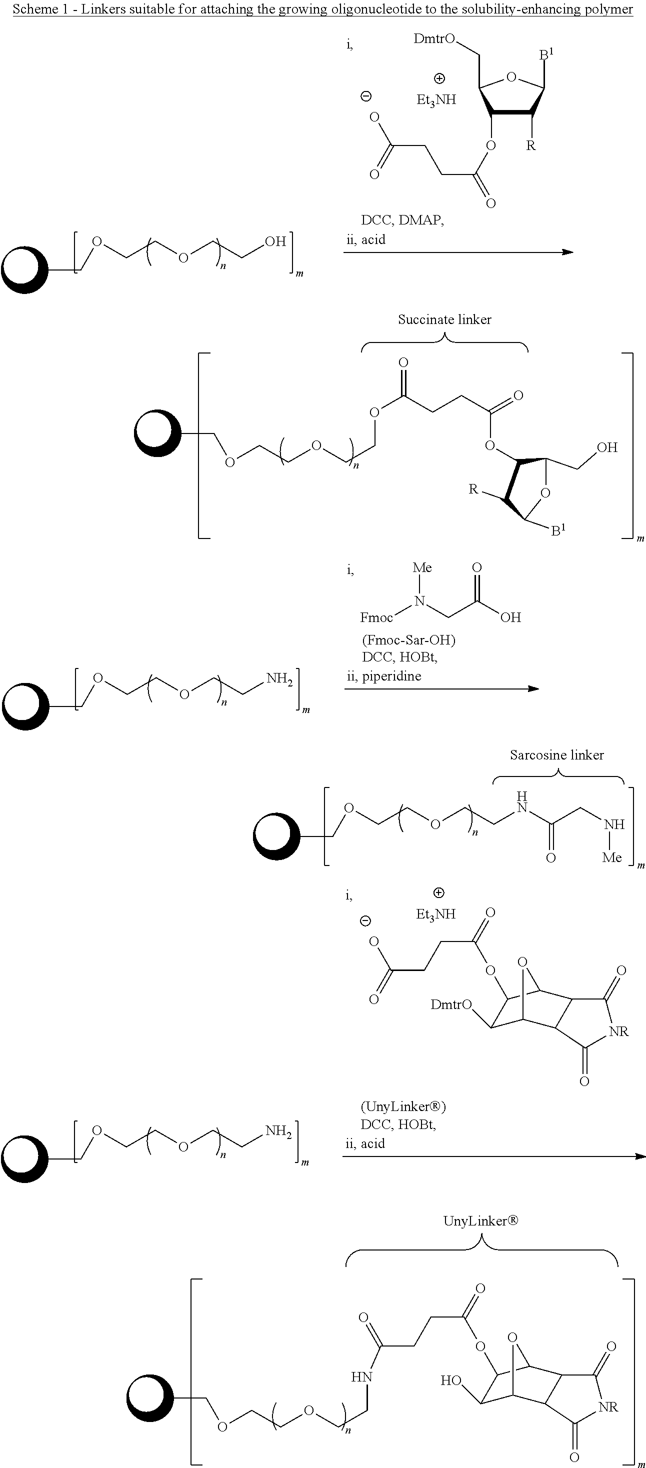

Alternatively, when the solution-phase process comprises preparation of an oligonucleotide via phosphoramidite chemistry, a PEG-amine may be reacted with one of the “universal” linkers. Universal linkers are desirable because the support can be loaded directly with a nucleoside phosphoramidite of choice (but liberating the hydroxy terminal oligo during global deprotection) without recourse to a separate nucleoside succinate building block. After loading onto the soluble synthesis support a temporary hydroxyl protecting group, usually Dmtr, is unblocked ready to participate in the oligo chain extension cycle.

Suitably, each growing oligonucleotide is attached at one of its ends to a solubility-enhancing polymer via a linker having a molecular weight of <600 Da. More suitably, the linker has a molecular weight of <300 Da.

The central hub may take a variety of forms. The central hub may be an atom (e.g., N or C) or an organic moiety (such as a benzene ring), onto which the one or more solubility-enhancing polymers are attached, directly or indirectly. Suitably, the central hub of each soluble synthesis support has a molecular mass of <1500 Da. Most suitably, the central hub of each soluble synthesis support has a molecular mass of <300 Da (e.g., a carbon atom).

The one or more solubility-enhancing polymers may be selected from the group consisting of poly(alkylene glycols), polyesters, polyamides, vinyl polymers, diene polymers, poly(alkylene imines), poly(amidoamines) and polysiloxanes. Examples of the solubility-enhancing polymer(s) include poly(ethylene glycol) (PEG), poly(propylene glycol) (PPG), poly(butylene glycol), poly(dimethylsiloxane) (PDMS), polybutadiene, polyisoprene, polystyrene, nylon, poly(ethylene imine) (PEI), poly(propylene imine), poly(L-lysine) (PLL), poly(methyl methacrylate) (PMMA), poly(vinyl benzoic acid), poly(hydroxystyrene), N-substituted glycines, and poly(lactide-co-glycolide) (PLGA). Suitably, the one or more solubility-enhancing polymers are selected from the group consisting of poly(alkylene glycols) (e.g., PEG), polyesters (e.g., poly(lactide-co-glycolide) and polysiloxanes (e.g., PDMS). Even more suitably, the one or more solubility-enhancing polymers are poly(alkylene glycols). In particular embodiments, the one or more solubility-enhancing polymers is PEG. PEG is highly soluble in acetonitrile, the solvent favoured by industry for coupling nucleotides to prepare oligonucleotides.

In an embodiment, each growing oligonucleotide is attached at one of its ends to a solubility-enhancing polymer via a linker having a molecular weight of <600 Da and the one or more solubility-enhancing polymers is PEG. In an embodiment, each growing oligonucleotide is attached at one of its ends to a solubility-enhancing polymer via a linker having a molecular weight of <300 Da and the one or more solubility-enhancing polymers is PEG.

The one or more solubility-enhancing polymers may be a PEG derivative, such as poly(propylene glycol), poly(ether amines) (e.g., Jeffamine® or Elastamine®) or (H₂NCHMeCH₂(OCHMeCH₂)ₓ(OCH₂CH₂)_{y}OMe), which are commercially available in a range of lengths (small to large x+y) and hydrophobicities (x/y large=hydrophobic; x/y small=hydrophilic).

In an embodiment, the central hub of each soluble synthesis support has a molecular mass of <1500 Da and the one or more solubility-enhancing polymers is PEG. In an embodiment, the central hub of each soluble synthesis support has a molecular mass of <300 Da (e.g., a carbon atom) and the one or more solubility-enhancing polymer is PEG.

The total molecular weight of the one or more solubility-enhancing polymers present within each molecule of soluble synthesis support is ≥1000 Da (e.g., ≥2000 Da). Suitably, the total molecular weight of the one or more solubility-enhancing polymers present within each molecule of soluble synthesis support is ≥4000 Da. As used herein, the molecular weight of a given solubility-enhancing polymer refers to the mass of the polymeric (i.e., repeating) portion of the polymer. By way of example, for a PEG solubility-enhancing polymer that is attached to the central hub at one end and to the growing oligonucleotide at the other end, the molecular weight of the solubility-enhancing polymer is the mass of all —[CH₂CH₂O]— repeating units. For illustrative purposes, each molecule of soluble synthesis support may contain a carbon atom (as central hub) attached directly or indirectly to 4 PEG polymers (serving as solubility-enhancing polymers), where each PEG polymer has a molecular weight of 2500 Da (approximately 57 repeating —[CH₂CH₂O]— units) (i.e., a 10 kDa 4-arm PEG star support). Suitably, the total molecular weight of the one or more solubility-enhancing polymers present within each molecule of soluble synthesis support is ≥8000 Da. More suitably, the total molecular weight of the one or more solubility-enhancing polymers present within each molecule of soluble synthesis support is ≥9000 Da. Yet more suitably, the total molecular weight of the one or more solubility-enhancing polymers present within each molecule of soluble synthesis support is ≥9500 Da. Most suitably, the total molecular weight of the one or more solubility-enhancing polymers present within each molecule of soluble synthesis support is ≥10,000 Da. Alternatively, the total molecular weight of the one or more solubility-enhancing polymers present within each molecule of soluble synthesis support is ≥15,000 Da. Suitably, the total molecular weight of the one or more solubility-enhancing polymers present within each molecule of soluble synthesis support is ≥20,000. More suitably, the total molecular weight of the one or more solubility-enhancing polymers present within each molecule of soluble synthesis support is ≥30,000 Da.

In an embodiment, the one or more solubility-enhancing polymer is PEG and the total molecular weight of the one or more solubility-enhancing polymers present within each molecule of soluble synthesis support is ≥9000 Da. In an embodiment, the one or more solubility-enhancing polymer is PEG and the total molecular weight of the one or more solubility-enhancing polymers present within each molecule of soluble synthesis support is ≥20,000.

The one or more solubility-enhancing polymers may be a single solubility-enhancing polymer or a plurality (e.g., 2-12) of solubility-enhancing polymers, each solubility-enhancing polymer having a molecular weight of ≥1000 Da. Suitably, each solubility-enhancing polymer has a molecular weight of ≥2000 Da. More suitably, each solubility-enhancing polymer has a molecular weight of ≥2250 Da. Alternatively, each solubility-enhancing polymer has a molecular weight of ≥4000 Da. Suitably, each solubility-enhancing polymer has a molecular weight of ≥8000 Da. More suitably, each solubility enhancing polymer has a molecular weight of ≥10,000 Da

The one or more solubility-enhancing polymers may be 2-10 solubility-enhancing polymers, each having a molecular weight of ≥1000 Da, or each having a molecular weight of ≥2000 Da, or each having a molecular weight of ≥2250 Da, or each having a molecular weight of ≥4000 Da, or each having a molecular weight of ≥8000 Da, or each having a molecular weight of ≥10,000 Da.

The one or more solubility-enhancing polymers may be 2-8 solubility-enhancing polymers, each having a molecular weight of ≥1000 Da, or each having a molecular weight of ≥2000 Da, or each having a molecular weight of ≥2250 Da, or each having a molecular weight of ≥4000 Da, or each having a molecular weight of ≥8000 Da, or each having a molecular weight of ≥10,000 Da.

The one or more solubility-enhancing polymers may be 3-4 solubility-enhancing polymers, each having a molecular weight of ≥1000 Da, or each having a molecular weight of ≥2000 Da, or each having a molecular weight of ≥2250 Da, or each having a molecular weight of ≥4000 Da, or each having a molecular weight of ≥8000 Da, or each having a molecular weight of ≥10,000 Da.

Each molecule of soluble synthesis support suitably may have a structure according to Formula I below:

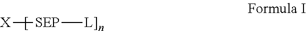

wherein
X represents the central hub (e.g., a carbon atom);
SEP represents a solubility-enhancing polymer (e.g., PEG);
L is absent or a linker (e.g., an organic moiety having a molecular weight of <600 Da or <300 Da); and
n is 2-12 (e.g., 2-10, 2-8 or 3-4).

Each growing oligonucleotide may be attached at one of its ends to L (when present), to SEP or to X. Suitably, each growing oligonucleotide is attached at one of its ends to L.

In an embodiment, the one or more solubility-enhancing polymers is 2-8 PEG polymers, each having a molecular weight of ≥1000 Da. In an embodiment, the one or more solubility-enhancing polymers is 3-4 PEG polymers, each having a molecular weight of ≥2250 Da (e.g., ≥2500 Da). In an embodiment, the one or more solubility-enhancing polymers is 2-8 PEG polymers, each having a molecular weight of ≥8000 Da.

In an embodiment, each molecule of soluble synthesis support comprises 4 PEG polymers, each having a molecular weight of 2300-2800 Da. Suitably, each molecule of soluble synthesis support comprises 4 growing oligonucleotides, each one being attached at one end to a PEG polymer. For example, the soluble synthesis support may be a 10 kDa 4-arm PEG star, a term used herein to denote a support comprising 4 PEG chains, each of 2500 Da, radiating out from a carbon atom acting as central hub.

In an embodiment, each molecule of soluble synthesis support comprises 4 PEG polymers, each having a molecular weight of 4000-6000 Da. Suitably, each molecule of soluble synthesis support comprises 4 oligonucleotides, each one being attached at one end to a PEG polymer. For example, the soluble synthesis support may be a 20 kDa 4-arm PEG star, a term used herein to denote a support comprising 4 PEG chains, each of 4000-6000 Da, radiating out from a carbon atom acting as central hub.

In an embodiment, each molecule of soluble synthesis support comprises 4 PEG polymers, each having a molecular weight of 8000-12,000 Da. Suitably, each molecule of soluble synthesis support comprises 4 growing oligonucleotides, each one being attached at one end to a PEG polymer. For example, the soluble synthesis support may be a 40 kDa 4-arm PEG star, a term used herein to denote a support comprising 4 PEG chains, each of 10 kDa, radiating out from a carbon atom acting as central hub.

The process may further comprise one or more deprotection steps. In particular, the process may comprise deprotecting the protected uracil nucleobase and/or the protected thymine nucleobase. It will be understood that deprotecting refers to removal of the protecting group(s) in the protected uracil and/or the protected thymine of the growing oligonucleotide. Any suitable deprotection technique may be used, such as ammonolysis, selective oxidation, selective reduction, oximate displacement, protecting group cleavage with an organometallic catalyst, protecting group cleavage with fluoride, protecting group cleavage with an acid, protecting group cleavage with a base, or a combination of one or more of these deprotection techniques. It may be that deprotecting the protected uracil nucleobase and/or the thymine nucleobase occurs during the step of growing the oligonucleotide and/or after the step of growing the oligonucleotide. Most suitably, deprotecting the protected uracil nucleobase and/or the thymine nucleobase occurs after the step of growing the oligonucleotide (i.e., deprotection is of the fully grown oligonucleotide).

In some embodiments, deprotecting the protected uracil nucleobase and/or the thymine nucleobase occurs after the step of growing the oligonucleotide and each resulting oligonucleotide (i.e., deprotected) has a molecular weight of ≥2000 Da. In some embodiments, deprotecting the protected uracil nucleobase and/or the thymine nucleobase occurs after the step of growing the oligonucleotide and each oligonucleotide (i.e., deprotected) has a molecular weight of ≥5000 Da.

It may be that the one or more deprotection step(s) does not occur until after the final coupling reaction. In such an embodiment, the oligonucleotide (once fully grown) may be protected (i.e., comprise a protecting group). Accordingly, the present invention also relates to a solution-phase process for the preparation of an oligonucleotide, wherein the oligonucleotide comprises at least one protected uracil nucleobase and/or at least one protected thymine nucleobase.

The oligonucleotide (once fully grown) may comprise uracil nucleobases wherein at least 10% of the uracil nucleobases are protected uracil nucleobases. The protected uracil nucleobases are as defined anywhere herein. Suitably, the oligonucleotide comprises uracil nucleobases wherein at least 30% of the uracil nucleobases are protected uracil nucleobases. More suitably, the oligonucleotide comprises uracil nucleobases wherein at least 50% of the uracil nucleobases are protected uracil nucleobases. Even more suitably, the oligonucleotide comprises uracil nucleobases wherein at least 70% of the uracil nucleobases are protected uracil nucleobases. Yet more suitably, the oligonucleotide comprises uracil nucleobases wherein at least 90% of the uracil nucleobases are protected uracil nucleobases. Yet even more suitably, the oligonucleotide comprises uracil nucleobases wherein at least 95% of the uracil nucleobases are protected uracil nucleobases. Yet still even more suitably, the oligonucleotide comprises uracil nucleobases wherein at least 99% of the uracil nucleobases are protected uracil nucleobases. Most suitably, the oligonucleotide comprises at least one uracil nucleobase wherein 100% (i.e., all) of the uracil nucleobase(s) are protected uracil nucleobases.

The oligonucleotide (once fully grown) may comprise thymine nucleobases wherein at least 10% of the thymine nucleobases are protected thymine nucleobases. The protected thymine nucleobases are as defined anywhere herein. Suitably, the oligonucleotide comprises thymine nucleobases wherein at least 30% of the thymine nucleobases are protected thymine nucleobases. More suitably, the oligonucleotide comprises thymine nucleobases wherein at least 50% of the thymine nucleobases are protected thymine nucleobases. Even more suitably, the oligonucleotide comprises thymine nucleobases wherein at least 70% of the thymine nucleobases are protected thymine nucleobases. Yet more suitably, the oligonucleotide comprises thymine nucleobases wherein at least 90% of the thymine nucleobases are protected thymine nucleobases. Yet even more suitably, the oligonucleotide comprises thymine nucleobases wherein at least 95% of the thymine nucleobases are protected thymine nucleobases. Yet still even more suitably, the oligonucleotide comprises thymine nucleobases wherein at least 99% of the thymine nucleobases are protected thymine nucleobases. Most suitably, the oligonucleotide comprises at least one thymine nucleobase wherein 100% (i.e., all) of the thymine nucleobase(s) are protected thymine nucleobases.

In an embodiment, the oligonucleotide (once fully grown) comprises thymine nucleobases and uracil nucleobases, wherein at least 10% of the thymine nucleobases are protected thymine nucleobases and at least 10% of the uracil nucleobases are protected uracil nucleobases. Suitably, the oligonucleotide comprises thymine nucleobases and uracil nucleobases, wherein at least 30% of the thymine nucleobases are protected thymine nucleobases and at least 30% of the uracil nucleobases are protected uracil nucleobases. More suitably, the oligonucleotide comprises thymine nucleobases and uracil nucleobases, wherein at least 50% of the thymine nucleobases are protected thymine nucleobases and at least 50% of the uracil nucleobases are protected uracil nucleobases. Even more suitably, the oligonucleotide comprises thymine nucleobases and uracil nucleobases, wherein at least 70% of the thymine nucleobases are protected thymine nucleobases and at least 70% of the uracil nucleobases are protected uracil nucleobases. Yet more suitably, the oligonucleotide comprises thymine nucleobases and uracil nucleobases, wherein at least 90% of the thymine nucleobases are protected thymine nucleobases and at least 90% of the uracil nucleobases are protected uracil nucleobases. Yet even more suitably, the oligonucleotide comprises thymine nucleobases and uracil nucleobases, wherein at least 95% of the thymine nucleobases are protected thymine nucleobases and at least 95% of the uracil nucleobases are protected uracil nucleobases. Yet still even more suitably, the oligonucleotide comprises thymine nucleobases and uracil nucleobases, wherein at least 99% of the thymine nucleobases are protected thymine nucleobases and at least 99% of the uracil nucleobases are protected uracil nucleobases. Most suitably, the oligonucleotide comprises at least one thymine nucleobase and at least one uracil nucleobase, wherein 100% (i.e., all) of the thymine nucleobase(s) are protected thymine nucleobases and 100% (i.e., all) of the uracil nucleobase(s) are protected uracil nucleobases.

Once the amount of protected uracil and/or protected thymine nucleobases has been determined in the oligonucleotide (once fully grown-after the final coupling reaction), one or more of the protected uracil and/or protected thymine nucleobases of the oligonucleotide may be deprotected. Deprotection is as defined anywhere herein. Suitably, all of the protected uracil and/or protected thymine nucleobases of the oligonucleotide are deprotected.

The process may further comprise the step of cleaving the oligonucleotide, once fully grown from the soluble synthesis support.

The oligonucleotide and/or the growing oligonucleotide may have at least one backbone modification, and/or at least one sugar modification and/or at least one base modification compared to an RNA or DNA-based oligonucleotide.

The oligonucleotide and/or the growing oligonucleotide may contain at least 1 modified nucleotide residue. The modification may be at the 2′ position of the sugar moiety. Sugar modifications in oligonucleotides/growing oligonucleotides described herein may include a modified version of the ribosyl moiety, such as 2′-O-modified RNA such as 2′-O-alkyl or 2′-O (substituted)alkyl e.g., 2′-O-methyl, 2′-O-(2-cyanoethyl), 2′-O-(2-methoxy)ethyl(2′-MOE), 2′-O-(2-thiomethyl)ethyl, 2′-O-butyryl, 2′-O-propargyl, 2′-O-allyl, 2′-O-(3-amino)propyl, 2′-O-(3-(dimethylamino)propyl), 2′-O-(2-amino)ethyl, 2′-O-(2-(dimethylamino)ethyl); 2′-deoxy (DNA); 2′-O (haloalkoxy)methyl (Arai K. et al. Bioorg. Med. Chem. 2011, 21, 6285) e.g., 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl] (MOCE), 2′-O-[2-(N-methylcarbamoyl)ethyl] (MCE), 2′-O-[2-(N,N-dimethylcarbamoyl)ethyl] (DCME); 2′-halo e.g. 2′-F, FANA (2′-F arabinosyl nucleic acid); carbasugar and azasuar modifications; 3′-O-alkyl e.g. 3′-O-methyl, 3′-O-butyryl, 3′-O-propargyl; and their derivatives.

Sugar modifications may be selected from the group consisting of 2′-fluoro (2′-F), 2′-O-methyl(2′-OMe), 2′-O-methoxyethyl(2′-MOE), and 2′-amino. Alternatively, the modification may be 2′-O-MOE. Other sugar modifications include “bridged” or “bicylic” nucleic acid (BNA), e.g. locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA), tricyclo DNA; unlocked nucleic acid (UNA); cyclohexenyl nucleic acid (CeNA), altritol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PPMO, PMOPlus, PMO-X); and their derivatives.

The oligonucleotides and/or the growing oligonucleotides may include other modifications, such as peptide-base nucleic acid (PNA), boron modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), oligonucleotides with integrated bases and backbones (ONIBs), pyrrolidine-amide oligonucleotides (POMs); and their derivatives.

The modified oligonucleotide and/or growing oligonucleotide may comprise a phosphorodiamidate morpholino oligomer (PMO), a locked nucleic acid (LNA), a peptide nucleic acid (PNA), a bridged nucleic acid (BNA) such as (5)-cEt-BNA, or a SPIEGELMER.

Modifications may also be present in the nucleobase. Base modifications include modified versions of the natural purine and pyrimidine bases (e.g. adenine, uracil, guanine, cytosine, and thymine), such as inosine, hypoxanthine, orotic acid, agmatidine, lysidine, 2-thiopyrimidine (e.g. 2-thiouracil, 2-thiothymine), G-clamp and its derivatives, 5-substituted pyrimidine (e.g. 5-methylcytosine, 5-methyluracil, 5-halouracil, 5-propynyluracil, 5-propynylcytosine, 5-aminomethyl uracil, 5-hydroxymethyl uracil, 5-aminomethylcytosine, 5-hydroxymethylcytosine, Super 5 T), 2,6-diaminopurine, 7-deazaguanine, 7-deazaadenine, 7-aza-2, 6-diaminopurine, 8-aza-7-deazaguanine, 8-aza-7-deazaadenine, 8-aza-7-deaza-2, 6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), or derivatives thereof; and degenerate or universal bases, like 2,6-difluorotoluene or absent bases like abasic sites (e.g. 1-deoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173. cPent-G, cPent-AP and Pr-AP were shown to reduce immunostimulatory effects when incorporated in SiRNA.¹¹

The nucleobase modification may be selected from the group consisting of 5-methyl pyrimidines, 7-deazaguanosines and abasic nucleotides. Alternatively, the modification may be a 5-methyl cytosine.

The oligonucleotides and/or growing oligonucleotides may include a backbone modification, e.g. a modified version of the phosphodiester present in RNA, such as phosphorothioate (PS), phosphorodithioate (PS2), phosphonoacetate (PACE), phosphonoacetamide (PACA), thiophosphonoacetate, thiophosphonoacetamide, phosphorothioate prodrug, H-phosphonate, methylphosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methylboranophosphonothioate, and their derivatives. Another modification includes phosphoramidite, phosphoramidate, N3′~PS' phosphoramidate, phosphordiamidate, phosphorothiodiamidate, sulfamate, dimethylenesulfoxide, sulfonate, triazole, oxalyl, carbamate, methyleneimino (MMI), and thioacetamido nucleic acid (TANA); and their derivatives.

Backbone modifications may be selected from the group consisting of: phosphorothioate (PS), phosphoramidate (PA) and phosphorodiamidate. The modified oligonucleotide may be a phosphorodiamidate morpholino oligomer (PMO). A PMO has a backbone of methylenemorpholine rings with phosphorodiamidate linkages. Suitably, the oligonucleotide and/or growing oligonucleotide may have a phosphorothioate (PS) backbone.

The oligonucleotide and/or growing oligonucleotide may comprise a combination of two or more modifications as described above. A person skilled in the art will appreciate that there are many synthetic derivatives of oligonucleotides.

The oligonucleotide and/or growing oligonucleotide may be a gapmer. The 5′ and 3′ wings of the gapmer may comprise or consist of 2′-MOE modified nucleotides. The gap segment of the gapmer may comprise or consist of nucleotides containing hydrogen at the 2′ position of the sugar moiety, i.e., is DNA-like. For example, the 5′ and 3′ wings of the gapmer may consist of 2′-MOE modified nucleotides and the gap segment of the gapmer may consist of nucleotides containing hydrogen at the 2′ position of the sugar moiety (i.e., deoxynucleotides). Alternatively, the 5′ and 3′ wings of the gapmer may consist of 2′-MOE modified nucleotides and the gap segment of the gapmer may consist of nucleotides containing hydrogen at the 2′ position of the sugar moiety (i.e., deoxynucleotides) and the linkages between all of the nucleotides are phosphorothioate linkages.

According to a second aspect of the present invention, there is provided an oligonucleotide obtained, directly obtained or obtainable by the process of the first aspect.

The following numbered statements 1 to 62 are not claims, but instead describe particular aspects and embodiments of the invention:
1. A solution-phase process for the preparation of an oligonucleotide, the process comprising the step of:
   growing the oligonucleotide by performing one or more sequential coupling reactions, each sequential coupling reaction increasing the length of the growing oligonucleotide by at least one nucleotide,
wherein the growing oligonucleotide comprises at least one protected uracil nucleobase and/or at least one protected thymine nucleobase, and
wherein the step of growing the oligonucleotide comprises one or more membrane filtration steps to isolate the growing oligonucleotide.
2. The solution-phase process of statement 1, wherein the growing oligonucleotide is a single growing oligonucleotide or a plurality (e.g., 2-12) of growing oligonucleotides.
3. The solution-phase process of statement 1 or 2, wherein the growing oligonucleotide is 2-10 growing oligonucleotides.
4. The solution-phase process of statement 1, 2 or 3, wherein the growing oligonucleotide is 2-8 growing oligonucleotides.
5. The solution-phase process of any one of the preceding statements, wherein the growing oligonucleotide is 3-4 growing oligonucleotides.
6. The solution-phase process of any one of the preceding statements, wherein each oligonucleotide has a molecular weight of ≥1000 Da.
7. The solution-phase process of any one of the preceding statements, wherein each oligonucleotide has a molecular weight of ≥2000 Da.
8. The solution-phase process of any one of the preceding statements, wherein each oligonucleotide has a molecular weight of ≥3000 Da.
9. The solution-phase process of any one of the preceding statements, wherein each oligonucleotide has a molecular weight of ≥5000 Da.
10. The solution-phase process of any one of the preceding statements, wherein the growing oligonucleotide comprises:
(i) at least one protected uracil nucleobase, wherein the protected uracil nucleobase comprises a protecting group; and/or
(ii) at least one protected thymine nucleobase, wherein the protected thymine nucleobase comprises a protecting group.
11. The solution-phase process of any one of the preceding statements, wherein the at least one protected uracil nucleobase comprises a protecting group and is attached to the growing oligonucleotide at the N1 position, the protecting group being located at the N3 position or the 04 position.
12. The solution-phase process of any one of the preceding statements, wherein the at least one protected thymine nucleobase comprises a protecting group and is attached to the growing oligonucleotide at the N1 position, the protecting group being located at the N3 position or the 04 position.
13. The solution-phase process of any one of the preceding statements, wherein the at least one protected uracil nucleobase comprises a protecting group and is attached to the growing oligonucleotide at the C5 position, the protecting group being located at the N3 position or the O2 position.
14. The solution-phase process of any one of statements 11, 12 or 13, wherein each protecting group is independently an acid-labile protecting group, a base labile protecting group, an ammonia labile protecting group, an oximate labile protecting group, an oxidatively labile protecting group, a hydrogenolytically labile protecting group or a transition metal catalysed cleavage protecting group.
15. The solution-phase process of any one of statements 11-14, wherein each protecting group is independently selected from the group consisting of 2,4,6-trimethylphenyl, 2-nitrophenyl, 2,4-dimethylphenyl, toluyl, 2-(4-nitrophenyl)ethyl, 2-(4-cyanophenyl)ethyl, allyl, benzoyl (Bz), 2,4-dimethylbenzoyl, tert-Bu-benzoyl (tert-BuBz) (e.g., p-tert-BuBz), acetyl (Ac), anisoyl (An) (e.g., p-An), 4-chlorobenzoyl, diphenylcarbamoyl, butylthiocarbonyl, 2-nitrophenylsulfenyl, 2,4-dinitrophenylsulfenyl, 2-nitro-4-toluylsulfenyl, and triphenylmethylsulfenyl.
16. The solution-phase process of any one of statements 11-15, wherein each protecting group is independently selected from the group consisting of benzoyl (Bz), 2,4-dimethylbenzoyl, tert-Bu benzoyl (tert-BuBz) (e.g., p-tert-BuBz) and anisoyl (An) (e.g., p-An).
17. The solution-phase process of any one of the preceding statements, wherein the step of growing the oligonucleotide comprises a single coupling reaction.
18. The solution-phase process of any one of the preceding statements, wherein the step of growing the oligonucleotide comprises performing two or more sequential coupling reactions.
19. The solution-phase process of any one of the preceding statements, wherein the step of growing the oligonucleotide comprises performing three or more sequential coupling reactions.
20. The solution-phase process of any one of the preceding statements, wherein the step of growing the oligonucleotide comprises performing four or more sequential coupling reactions.
21. The solution-phase process of any one of the preceding statements, wherein the step of growing the oligonucleotide comprises performing six or more sequential coupling reactions.
22. The solution-phase process of any one of the preceding statements, wherein the step of growing the oligonucleotide comprises performing ten or more sequential coupling reactions.
23. The solution-phase process of any one of the preceding statements, wherein the step of growing the oligonucleotide comprises performing fifteen or more sequential coupling reactions.
24. The solution-phase process of any one of the preceding statements, wherein each sequential coupling reaction increases the length of the growing oligonucleotide by one nucleotide.
25. The solution-phase process of any one of the preceding statements, wherein the step of growing the oligonucleotide is conducted in at least one organic solvent.
26. The solution-phase process of any one of the preceding statements, wherein the step of growing the oligonucleotide is conducted in acetonitrile, optionally mixed with another organic solvent.
27. The solution-phase process of any one of the preceding statements, wherein the step of growing the oligonucleotide is conducted in acetonitrile mixed with sulfolane (e.g., 4:1 v/v).
28. The solution-phase process of any one of statements 1-26, wherein the step of growing the oligonucleotide is conducted in acetonitrile (i.e., neat acetonitrile).
29. The solution-phase process of any one of the preceding statements, wherein the step of growing the oligonucleotide is conducted in neat acetonitrile or acetonitrile mixed with sulfolane (e.g., 4:1 v/v).
30. The solution-phase process of any one of the preceding statements, wherein the membrane filtration is membrane diafiltration.
31. The solution-phase process of any one of the preceding statements, wherein the membrane filtration is organic solvent nanofiltration (OSN) or ultrafiltration (UF).
32. The solution-phase process of any one of the preceding statements, wherein membrane filtration is performed once or twice per coupling reaction.
33. The solution-phase process of any one of the preceding statements, wherein membrane filtration is performed as part of every sequential coupling reaction conducted in the step of growing the oligonucleotide.
34. The solution-phase process of any one of the preceding statements, wherein the step of growing the oligonucleotide and the one or more membrane filtration steps to isolate the growing oligonucleotide are conducted in the same solvent.
35. The solution-phase process of any one of the preceding statements, wherein membrane filtration is performed with a membrane formed from or comprises a polymeric material suitable for fabricating microfiltration, ultrafiltration, nanofiltration or reverse osmosis membranes, including polyethylene, polypropylene, polytetrafluoroethylene (PTFE), polyvinylidene difluoride (PVDF), polysulfone, polyethersulfone, polyacrylonitrile, polyamide, polyester, polyimide, polyetherimide, cellulose acetate, polyaniline, polypyrrole, polybenzimidazole, polyetheretherketone (PEEK) and mixtures thereof.
36 The solution-phase process of any one of the preceding statements, wherein membrane filtration is performed using a crosslinked polybenzimidazole membrane (e.g., an integrally skinned, asymmetric, crosslinked polybenzimidazole membrane) or a polyetheretherketone membrane.
37. The solution-phase process of any one of the preceding statements, wherein the growing oligonucleotide is attached at one end to a soluble synthesis support.
38. The solution-phase process of statement 37, wherein the soluble synthesis support comprises a central hub and one or more solubility-enhancing polymers, each attached to the central hub.
39. The solution-phase process of statement 38, wherein the one or more solubility-enhancing polymers is a single solubility-enhancing polymer or a plurality (e.g., 2-12) of solubility-enhancing polymers.
40. The solution-phase process of statement 38 or 39, wherein the one or more solubility-enhancing polymers is 2-10 solubility-enhancing polymers.
41. The solution-phase process of statement 38, 39 or 40, wherein the one or more solubility-enhancing polymers is 2-8 solubility-enhancing polymers.
42. The solution-phase process of any one of statements 38-41, wherein the one or more solubility-enhancing polymers is 3-4 solubility-enhancing polymers.
43. The solution-phase process of any one of statements 38-42, wherein the central hub is an atom (e.g., N or C) or an organic moiety (such as a benzene ring), onto which the one or more solubility-enhancing polymers are attached, directly or indirectly.
44. The solution-phase process of any one of statements 38-43, wherein the central hub of each soluble synthesis support has a molecular mass of <1500 Da (e.g., <300 Da).
45. The solution-phase process of any one of statements 38-44, wherein the one or more solubility-enhancing polymers is selected from the group consisting of poly(alkylene glycols), polyesters, polyamides, vinyl polymers, diene polymers, poly(alkylene imines), poly(amidoamines) and polysiloxanes.
46 The solution-phase process of any one of statements 38-45, wherein the one or more solubility-enhancing polymers is selected from the group consisting of poly(ethylene glycol) (PEG), poly(propylene glycol) (PPG), poly(butylene glycol), poly(dimethylsiloxane) (PDMS), polybutadiene, polyisoprene, polystyrene, nylon, poly(ethylene imine) (PEI), poly(propylene imine), poly(L-lysine) (PLL), poly(methyl methacrylate) (PMMA), poly(vinyl benzoic acid), poly(hydroxystyrene), N-substituted glycines, and poly(lactide-co-glycolide) (PLGA).
47. The solution-phase process of any one of statements 38-46, wherein the one or more solubility-enhancing polymers is PEG.
48. The solution-phase process of any one of statements 38-47, wherein the total molecular weight of the one or more solubility-enhancing polymers present within each molecule of soluble synthesis support is ≥1000 Da, ≥2000 Da, ≥4000 Da, ≥8000 Da, ≥9000 Da, ≥9500 Da, ≥10,000 Da, ≥15,000 Da, ≥20,000 or ≥30,000 Da.
49. The solution-phase process of any one of statements 38-48, wherein each solubility-enhancing polymer has a molecular weight of ≥1000 Da, ≥2000 Da, ≥2250 Da, ≥4000 Da, ≥8000 Da, or ≥10,000 Da.
50. The solution-phase process of any one of statements 38-49, wherein each molecule of soluble synthesis support has a structure according to Formula I below:

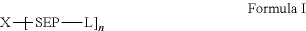

wherein
X represents the central hub;
SEP represents a solubility-enhancing polymer;
L is absent or a linker; and
n is 2-12 (e.g., 2-10, 2-8 or 3-4).
51. The solution-phase process of any one of the preceding statements, wherein the process further comprises one or more deprotection steps.
52. The solution-phase process of statement 51 wherein the one or more deprotection steps comprises selective oxidation, selective reduction, oximation, protecting group cleavage with an organometallic catalyst, protecting group cleavage with fluoride, protecting group cleavage with an acid, protecting group cleavage with a base, or a combination thereof.
53. The solution-phase process of statement 51 or 52, wherein the one or more deprotecting steps occurs during the step of growing the oligonucleotide and/or after the step of growing the oligonucleotide.
54. The solution-phase process of statement 51, 52 or 53, wherein the one or more deprotection steps occurs after the step of growing the oligonucleotide.
55. The solution-phase process of any one of the preceding statements, wherein the oligonucleotide comprises at least one protected uracil nucleobase and/or at least one protected thymine nucleobase.
56. The solution-phase process of statement 55, wherein the oligonucleotide comprises uracil nucleobases wherein at least 10%, 30%, 50%, 70%, 90%, 95%, 99% or 100% of the uracil nucleobases are protected uracil nucleobases.
57. The solution-phase process of statement 55 or 56, wherein the oligonucleotide comprises thymine nucleobases, wherein at least 10%, 30%, 50%, 70%, 90%, 95%, 99% or 100% of the thymine nucleobases are protected thymine nucleobases.
58. The solution-phase process of statement 55, 56 or 57, wherein one or more of the protected uracil and/or protected thymine nucleobases of the oligonucleotide undergoes a deprotection step.
59. The solution-phase process of any one of statements 55-58, wherein all of the protected uracil and/or protected thymine nucleobases of the oligonucleotide undergo a deprotection step.
60. The solution-phase process of statement 58 or 59, wherein the deprotection step comprises selective oxidation, selective reduction, oximation, protecting group cleavage with an organometallic catalyst, protecting group cleavage with fluoride, protecting group cleavage with an acid, protecting group cleavage with a base, or a combination thereof.
61. The solution-phase process of any one of statements 38-50, wherein the process further comprises the step of cleaving the oligonucleotide from the soluble synthesis support.
62. An oligonucleotide obtained, directly obtained or obtainable by the solution-phase process of any one of the preceding claims.

### EXAMPLES

One or more examples of the invention will now be described, for the purpose of illustration only, with reference to the accompanying figures:

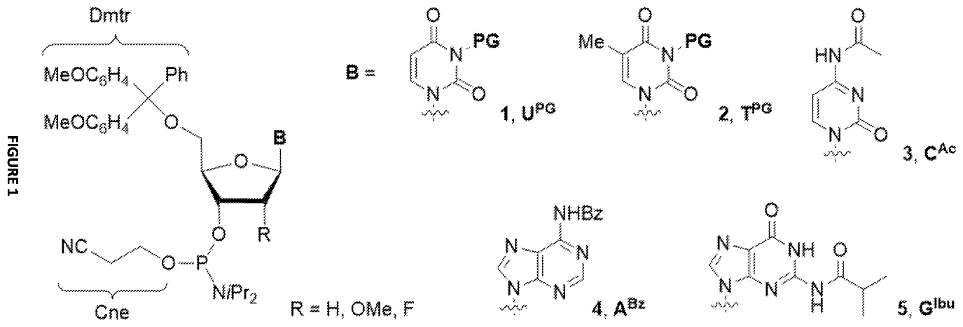

FIG. 1. shows the protected phosphoramidite structures which were assessed for solubility in acetonitrile.

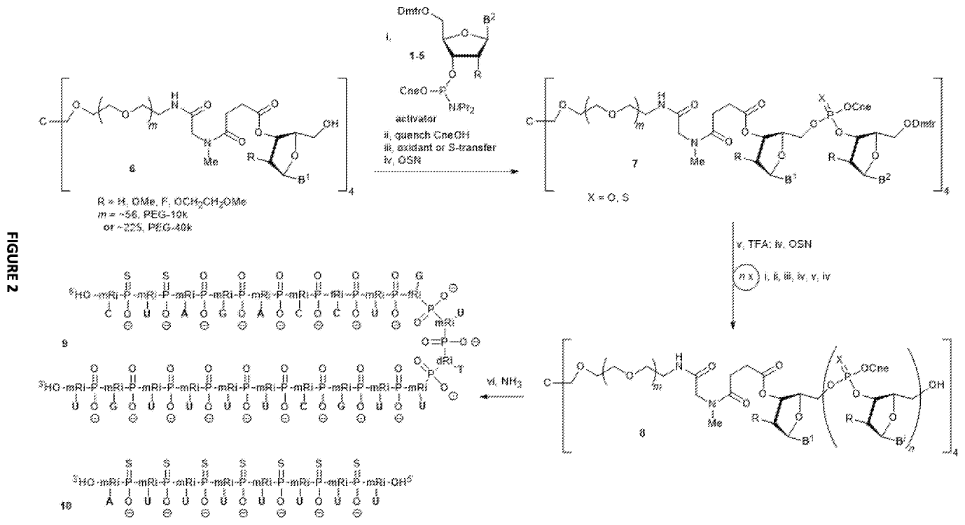

FIG. 2. shows the synthesis of oligos on a 10 kDa poly-disperse 4-arm star and a 40 kDa poly-disperse 4-arm star.

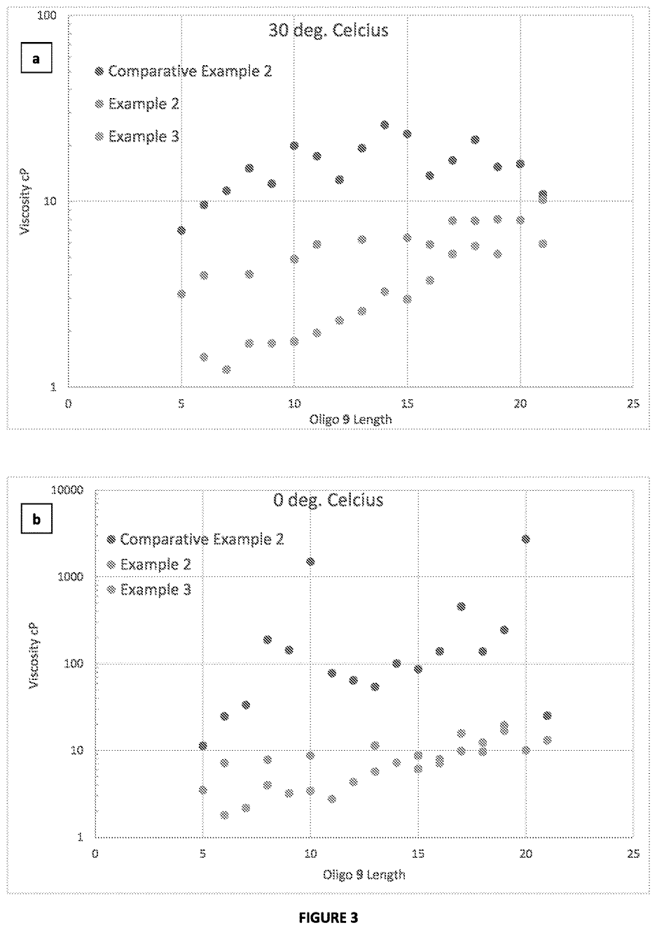

FIG. 3. shows viscosity measurements for comparative example 2 and examples 2 and 3 at: a) 30° C.; and b) 0° C. Measurements were taken from HO-pentamer-PEG star 8 onwards during growth of the oligo.

### Example 1

A range of 5′-O-Dmtr-nucleoside-3′-O-(cyanoethyl-N, N-diisopropyl) phosphoramidites (FIG. 1. compounds 1-5) were studied to determine their suitability for solution-phase oligonucleotide synthesis in acetonitrile. Each compound (ca. 0.5 g) was placed in a flask with a magnetic stirrer immersed in an oil bath held at 30° C. To the flask was added acetonitrile (0.200 mL) and the contents stirred for 2 min. After this, further aliquots of acetonitrile (0.050 mL) were added, each time followed by stirring for 2 min. The volume of acetonitrile required to establish a clear solution was recorded. The solubility so determined is recorded in Table 1.

TABLE 1
Properties of compounds 1-5 in acetonitrile

| Compound | | Protecting | | Solubility, | Solubility, |
|---|---|---|---|---|---|
| No. | Base, B | group, PG | R | g/L | mol/L |
| 1a | U | None | OMe | 0.05 | 0.07 |
| 1b | U | Bz | OMe | 1.66 | 1.92 |
| 1c | U | p-tert-BuBz | OMe | 1.60 | 1.74 |
| 1d | U | p-An | OMe | 2.17 | 2.42 |
| 2a | T | None | H | 0.86 | 1.16 |
| 2b | T | Bz | H | 1.30 | 1.53 |
| 3 | C | Ac | OMe | 1.04 | 1.30 |
| 4 | A | Bz | OMe | 1.07 | 1.21 |
| 5 | G | Ibu | OMe | 1.11 | 1.28 |

Compound 1a stands out for its low solubility. Whilst 1a only achieves 0.05 M solubility, all of the other compounds exhibit greater than 1 M solubility in acetonitrile. After 1a (unprotected U derivative), the closely related unprotected T derivative (compound 2a) is the least soluble of the remaining building blocks 2 to 5. A series of N3-acylated derivatives were prepared from 5′-Dmtr-mU (Peyrat and Xie, “Synthesis of Thymidine Dimers from 5′-O-Aminothymidine”, Synthesis, 2012, 44, 1718-1724) and the intermediate nucleosides converted to the corresponding phosphoramidites 1b-d with chloro-2-cyanoethoxy-N,N-diisopropylaminophosphine (Sproat et al., New and Convenient Protection System for Pseudouridine, Highly Suitable for Oligoribonucleotide Synthesis”, J. Chem. Soc., Perkin Trans. 1, 1994, 3423-3429). These species ranged from approx. 25 to 35 times more soluble than compound 1a, with unprotected N3. Even for the more soluble unprotected T phosphoramidite, 2a, protection of N3 (compound 2b) increased the solubility by more than 30%. It is theorised that this increase in solubility of the pyrimidine building blocks 1 and 2 engendered by N3 protection is extended to protected oligos containing them.

### Comparative Example 2

FIG. 2 shows 21-mer sequence 9 which was selected as a test sequence for the effects of protecting U and T because of its unusually high proportion of 2′-methyl uridine (mU) residues. Sequence 9 was also found to give extremely viscous solutions when supported on a large soluble PEG-star (vide infra), which makes organic solvent nanofiltration (OSN) impractical due to seriously reduced mass transfer efficiency and the difficulty of pumping thick liquids.

Soluble synthesis support PEG-40k(SarH)₄ (m ~255) was condensed with Dmtr-mU succinate (2.5 eq. per arm) using dicyclohexyl carbodiimide (DCC) and hydroxybenzotriazole (HOBt). The solution was transferred with filtration into a single stage membrane separation synthesiser fitted with 5 circular cells (52 cm² each) of PBI16-DBX-M2005 membranes, reaching a final oligo concentration of 10 mM. The crude material was diafiltered in neat acetonitrile to remove low MW debris, permeating 4 system volumes (or diavolumes, DV) of solvent with the system maintained at 30° C. Detritylation was then performed within the synthesiser using 2.5% trifluoroacetic acid (TFA) and excess cation trap, e.g., dodecanethiol. The detritylation was quenched with excess 3-picoline, and diafiltration was resumed in neat acetonitrile (6 DV, final permeate flux 10 mL/min) until no remaining succinate building block could be detected to give pure PEG-40k(SarSuc-mU-OH)₄, 6, B¹=mU, m ~225.

Loaded nucleoside-star 6 was subjected to one cycle of chain extension: Nucleoside phosphoramidite 5 (R=MeO, 2 eq. per arm) was added to the synthesiser and activated with DCI, before being quenched with CneOH after 20 min. After 2 min, oxidation was initiated with excess camphor sulfonyl oxaziridine (CSO) in neat acetonitrile, the reaction proceeding for 1 hr. Upon completion, low MW reagent debris was removed by diafiltraton (4 DV), and the temporary 5′-Dmtr protecting group was unblocked as above with 2.5% TFA (thermostatted 30° C.). The detritylation was quenched with excess 3-picoline, and diafiltration continued (6 DV, final permeate flux 10 mL/min) until no building block debris remained.

This cycle was repeated, except that only 1.5 eq. phosphoramidite was used per cycle from trimer onwards to the end of the run. Reagents were injected into the synthesiser in the order required to build up sequence 9, removing excess reagents and debris by OSN as above. For this Comparative Example, unprotected mU 1a and dT 2a building blocks were used at the appropriate chain extensions. From hydroxy-pentamer-PEG-star onwards (FIG. 2, compound 8, n=4) the viscosity of the solution was determined immediately before new phosphoramidite was added at both 30° C. (normal synthesiser running temperature) and 0° C. Typically, at this is the point in the cycle, after maximum diafiltration, the lowest concentrations of contaminants are present.

As the length of the oligo on the PEG-star support increased, the permeate flux dropped slowly (from 14 mL/min at trimer) and the viscosity rose (FIG. 3). At 11-mer-star, the viscosity of the retentate and the rejection of the building block both rose such that it became difficult to proceed further, although the total permeate flux was still manageable (7.4 mL/min). Consequently, the solvent was exchanged for acetonitrile-sulfolane 4:1 v/v. Although mixtures of acetonitrile-sulfolane are more viscous than neat acetonitrile, the added sulfolane reduces the overall viscosity of longer oligo-PEG-star solutions. The chain extension cycles were resumed, but at 17-mer-star the permeate flow was reduced to an impractical level (2.4 mL/min), correlating with the rising viscosity. Therefore, the membrane was switched to a looser PBI14-DBX-M2005, which increased the permeate flow marginally at 18-mer (4.4 mL/min), so the run was continued with difficulty up to full-length 21-mer. For chain extensions 20 and 21 the CSO was replaced with xanthane hydride (XH) dissolved in pyridine to effect sulfur transfer, proceeding to diafiltration after 5 minutes.

After detritylation of the 21-mer-star, the solvent was exchanged for acetonitrile-pyridine 9:1 v/v; oligo-stars are highly soluble in pyridine and a corresponding large drop in viscosity was observed (FIG. 3). Notably, if this full-length 21-mer-PEG-star is dispersed in neat acetonitrile, it exhibits a viscosity of ca. 530 cP. The final oligo-star was washed from the synthesiser, and the solvent evaporated under reduced pressure. The crude oligo-star was deprotected in a mixture of conc. ammonia and 3 vol % diethylamine (DEA) at 35° C. for 18 hr. The following day the solvent was evaporated, then co-evaporated from acetonitrile (×3), and the full-length 21-mer 9 was isolated by trituration with acetonitrile in 44% UV-purity.

### Example 2

Soluble synthesis support PEG-10k(SarH)₄ was condensed with Dmtr-mU succinate (2.5 eq. per arm) using DCC and HOBt. The solution was transferred with filtration into a single membrane separation stage synthesiser fitted with 5 circular cells (52 cm² each) of PBI17-DBX-M2005 membranes, reaching a final oligo concentration of 20 mM-twice that in Comparative Example 2. The crude material was diafiltered in acetonitrile-sulfolane 4:1 v/v to remove low MW debris, permeating 4 DV of solvent with the system maintained at 30° C. Detritylation was then performed within the synthesiser using 2.5% TFA and excess cation trap, e.g., dodecanethiol. The detritylation was quenched with excess 3-picoline, and diafiltration was resumed in acetonitrile-sulfolane 4:1 v/v (6 DV, final flux 19 mL/min) until no remaining succinate building block could be detected to give pure PEG-10k(SarSuc-mU-OH)₄, 6, B¹=mU, m~56.

Loaded nucleoside-star 6 was subjected to one cycle of chain extension: Nucleoside phosphoramidite 5 (R=MeO, 2 eq. per arm) was added to the synthesiser and activated with DCI, before being quenched with CneOH after 20 min. After 2 min, oxidation was initiated with excess CSO in neat acetonitrile, the reaction proceeding for 1 hr. Upon completion, low MW reagent debris was removed by diafiltraton (4 DV), and the temporary 5′-Dmtr protecting group was unblocked as above with 2.5% TFA (thermostatted 30° C.). The detritylation was quenched with excess 3-picoline, and diafiltration continued (6 DV, final flux 17 mL/min) until no building block debris remained, using acetonitrile-sulfolane 4:1 v/v throughout.

This cycle was repeated, except that only 1.5 eq. phosphoramidite was used per cycle from trimer onwards to the end of the run. Reagents were injected into the synthesiser in the order required to build up sequence 9, removing excess reagents and debris by OSN as above. For this example, N3-protected mU^{Bz} 1b and dT^{Bz} 2b building blocks were used at the appropriate chain extensions. Only at 14-mer-star did a drop in permeate flow (5.8 mL/min) and a rise in building block rejection become apparent. The reaction proceeded following replacement of the membrane disks (8.2 mL/min at 15-mer to 6.4 mL/min at 21-mer). Sulfur transfer to the 20-mer and 21-mer oligo-stars was achieved with an acetonitrile solution of 3-phenyl 1,2,4-dithiazoline-5-one over 5 minutes.

From hydroxy-pentamer-PEG-star onwards (FIG. 2, compound 8, m=4) the viscosity of the solution was determined immediately before new phosphoramidite was added at both 30° C. (normal synthesiser running temperature), and 0° C. Unlike in Comparative Example 2, the viscosity remained low throughout the run, slowly rising to eventually reach just 10 cP (FIG. 3a). This was surprising because, not only was a much smaller solubilising PEG-star used in this example (10 kDa versus 40 kDa in Comparative Example 2), but also the oligo concentration had been doubled. Indeed, even chilling to 0° C. only led to a small rise in viscosity (FIG. 3b), meaning that the synthesiser could be cooled well below room temperature if necessary.

After detritylation of the 21-mer-star, the solvent was exchanged for acetonitrile-pyridine 9:1 v/v and the final oligo-star was washed from the synthesiser. The solvent was then evaporated under reduced pressure. The crude oligo-star was deprotected in a mixture of conc. ammonia and 3 vol % DEA at 35° C. for 18 hr. The following day the solvent was evaporated, then co-evaporated from acetonitrile (×3), and the full-length 21-mer 9 was isolated by trituration with acetonitrile in 64% UV-purity.

### Example 3

Soluble synthesis support PEG-10k(SarH)₄ was condensed with Dmtr-mU succinate (2.5 eq. per arm) using DCC and HOBt. The solution was transferred with filtration into a single membrane separation stage synthesiser fitted with 5 circular cells (52 cm² each) of PBI17-DBX-M2005 membranes, reaching a final oligo concentration of 20 mM. The crude material was diafiltered in neat acetonitrile (cf. acetonitrile-sulfolane 4:1 v/v in Example 2) to remove low MW debris, permeating 4 DV of solvent with the system maintained at 30° C. Detritylation was then performed within the synthesiser using 2.5% TFA and excess cation trap, e.g., dodecanethiol. The detritylation was quenched with excess 3-picoline, and diafiltration was resumed in neat acetonitrile (6 DV, final permeate flux 20 mL/min) until no remaining succinate building block could be detected to give pure PEG-10k(SarSuc-mU-OH)₄, 6, B¹=mU, m~56.

Loaded nucleoside-star 6 was subjected to one cycle of chain extension: Nucleoside phosphoramidite 5 (R=MeO, 2 eq. per arm) was added to the synthesiser and activated with DCI, before being quenched with CneOH after 20 min. After 2 min, oxidation was initiated with excess CSO in neat acetonitrile, the reaction proceeding for 1 hr. Upon completion, low MW reagent debris was removed by diafiltraton (4 DV), and the temporary 5′-Dmtr protecting group was then unblocked as above with 2.5% TFA (thermostatted 30° C.). The detritylation was quenched with excess 3-picoline, and diafiltration continued (6 DV, final flux 17 mL/min) until no building block debris remained, using neat acetonitrile throughout.

This cycle was repeated, except that only 1.5 eq. phosphoramidite was used per cycle from trimer onward to the end of the run. Reagents were injected into the synthesiser in the order required to build up sequence 9, removing excess reagents and debris by OSN as above. For this example, N3-protected mU^{Bz} 1b and dT^{Bz} 2b building blocks were used at the appropriate chain extensions. Only at 13-mer-star did a drop in permeate flow (6.5 mL/min) and a rise in building block rejection become apparent. The reaction proceeded following replacement of the membrane disks (10 mL/min at 14-mer to 5.1 mL/min at 21-mer). Sulfur transfer to the 20-mer and 21-mer oligo-stars was achieved with XH in pyridine over 5 minutes.

From hydroxy-pentamer-PEG-star onwards (FIG. 2, compound 8, m=4) the viscosity of the solution was determined immediately before new phosphoramidite was added at both 30° C. (normal synthesiser running temperature), and 0° C. At 30° C., during the early chain extension cycles the viscosity was significantly lower than in a solution of acetonitrile-sulfolane 4:1 (FIG. 3a), but as the length grew the viscosity began to approach that of Example 2. Upon chilling to 0° C. the difference in viscosity between Examples 2 and 3 was not so marked (FIG. 3b).

After detritylation and diafiltration of the 21-mer-star, the final oligo-star was washed from the synthesiser and the solvent evaporated under reduced pressure. The crude oligo-star was deprotected in a mixture of conc. ammonia and 3 vol % DEA at 35° C. for 18 hr. The following day the solvent was evaporated, then co-evaporated from acetonitrile (×3), and the full-length 21-mer 9 was isolated by trituration with acetonitrile in 54% UV-purity.

### Example 4

Soluble synthesis support PEG-10k(SarH)₄ was condensed with Dmtr-mA^{Bz} succinate (2.5 eq. per arm) using DCC and HOBt. The solution was transferred with filtration into a single membrane separation stage synthesiser fitted with 5 circular cells (52 cm² each) of PBI17-DBX-M2005 membranes, reaching a final oligo concentration of 10 mM. The crude material was diafiltered in neat acetonitrile to remove low MW debris, permeating 4 DV of solvent with the system maintained at 30° C. Detritylation was then performed within the synthesiser using 2.5% TFA and excess cation trap, e.g., dodecanethiol. The detritylation was quenched with excess pyridine, and diafiltration was resumed in neat acetonitrile (6 DV, final permeate flux 20 mL/min) until no remaining succinate building block could be detected to give pure PEG-10k(SarSuc-mA^{Bz}-OH)₄, 6, B¹=A^{Bz}, m~56.

Loaded nucleoside-star 6 was then subjected to chain extension: Nucleoside phosphoramidite 1b or 1c (B²=U^{Bz} or U^{tBuBz}, R=MeO, 2 eq. per arm) was added to the synthesiser and activated with DCI, before being quenched with CneOH after 20 min. After 2 min sulfur transfer was initiated with excess XH in pyridine, the reaction proceeding for 20 min. Upon completion, low MW reagent debris was removed by diafiltraton (4 DV), and the temporary 5′-Dmtr protecting group was unblocked as above with 2.5% TFA (thermostatted 30° C.). The detritylation was quenched with excess 3-picoline, and diafiltration continued (6 DV, final flux 17 mL/min) until no building block debris remained, using acetonitrile-sulfolane 4:1 v/v throughout.

This cycle was repeated, continuing with phosphoramidites 1b or 1c, to build the sequence mA(mU)₇ 10, except that only 1.5 eq. was used per cycle from trimer onwards to the end of the run, removing excess reagents and debris by OSN as above. In both cases, the oligo-star, PEG-10k[mA^{Bz}(mU^{PG})₇]₄, PG=Bz or tBuBz, remained in solution throughout the synthesis and the viscosity remained low. After detritylation and diafiltration of the 8-mer-star, the final oligo-star was washed from the synthesiser and the solvent evaporated under reduced pressure. The crude oligo-star was initially treated with DEA-DMF (3:7 v/v) and the solvent evaporated. The residue was then redissolved in conc. ammonia and heated in a sealed tube at 55° C. for 18 hr. The following day the solvent was evaporated, then co-evaporated from acetonitrile (×3), and the full-length 8-mer 10 was isolated by trituration with acetonitrile. The 8-mer prepared with mU^{Bz} phosphoramidite 1b had a UV-purity of 75%; the 8-mer prepared with mU^{tBuBz} phosphoramidite 1c had a UV-purity of 88%.

While specific embodiments of the invention have been described herein for the purpose of reference and illustration, various modifications will be apparent to a person skilled in the art without departing from the scope of the invention as defined by the appended claims.

### REFERENCES

1. U.S. Pat. No. 8,664,357
2. US9, 127, 123
3. U.S. Pat. No. 10,239,996
4. EP3347402
5. P. R. J. Gaffney, J. F. Kim, I. B. Valtcheva, G. D. Williams, M. S. Anson, A. M. Buswell, A. G. Livingston, Liquid-Phase Synthesis of 2′-Methyl-RNA on a Homostar Support through Organic-Solvent Nanofiltration, Chem. Eur. J., 2015, 21, 9535-9543
6. J. F. Kim, P. R. J. Gaffney, I. B. Valtcheva, G. Williams, A. M. Buswell, M. S. Anson, A. G. Livingston, Organic Solvent Nanofiltration (OSN): A New Technology Platform for Liquid-Phase Oligonucleotide Synthesis (LPOS), Org. Process Res. Dev. 2016, 20, 1439-1452
7. So Su, Peeva L. G., Tate E. W., Leatherbarrow R. J., Livingston A. G. “Organic Solvent Nanofiltration-A New Paradigm in Peptide Synthesis” Org. Process. Res. & Dev. 14 (2010) pp. 1313-132
8. Yeo J, Peeva L, Chung S, Gaffney P, Kim D, Luciani C, Tsukanov S, Siebert K, Kopach M, Albericio F, Livingston A “Liquid Phase Peptide Synthesis by One Pot Nanostar Sieving (PEPSTAR)”; Angew. Chem. Int. Ed. 2021, 60, pp 7786-7795
9. PEG and derivatives of PEG have also been synthesised using membrane separation, as described by Dong R., Liu R., Gaffney P. R. J., Schaepertoens M., Marchetti P., Williams C. M., Chen R. and Livingston A. G. “Sequence-defined multifunctional polyethers via liquid-phase synthesis with molecular sieving” Nature Chemistry (2019) 11 pp. 136-145
10. WO 2016/188835 A1
11. Peacock H. et al. J. Am. Chem. Soc. (2011), 133, 9200
12. Baran et al. Unlocking P(V): Reagents for chiral phosphorothioate synthesis, Science, Vol 361, 1234-1238. DOI: 10.1126/science.aau3369

1. A solution-phase process for the preparation of an oligonucleotide, the process comprising the step of:
growing the oligonucleotide by performing one or more sequential coupling reactions, each sequential coupling reaction increasing the length of the growing oligonucleotide by at least one nucleotide,
wherein the growing oligonucleotide comprises at least one protected uracil nucleobase and/or at least one protected thymine nucleobase, and
wherein the step of growing the oligonucleotide comprises one or more membrane filtration steps to isolate the growing oligonucleotide.

2. The solution-phase process of claim 1, wherein the oligonucleotide has a molecular weight of ≥1000 Da.

3. (canceled)

4. The solution-phase process of claim 1, wherein the growing oligonucleotide is attached at one end to a soluble synthesis support.

5. The solution-phase process of claim 4, wherein the soluble synthesis support comprises a central hub and one or more solubility-enhancing polymers, each attached to the central hub.

6. The solution-phase process of claim 5, wherein the one or more solubility-enhancing polymers is selected from the group consisting of poly(alkylene glycols), polyesters, polyamides, vinyl polymers, diene polymers, poly(alkylene imines), poly(amidoamines) and polysiloxanes.

7. The solution-phase process of claim 5, wherein the one or more solubility-enhancing polymers are poly(alkylene glycols).

8. The solution-phase process of claim 5, wherein the total molecular weight of the one or more solubility-enhancing polymers present within each molecule of soluble synthesis support is ≥4000 Da.

9. (canceled)

10. The solution-phase process of claim 5, wherein the one or more solubility-enhancing polymers is a single solubility-enhancing polymer or a plurality of solubility-enhancing polymers.

11. The solution-phase process of claim 10, wherein each solubility-enhancing polymer has a molecular weight of ≥1000 Da.

12. The solution-phase process of claim 1, wherein the step of growing the oligonucleotide comprises performing two or more sequential coupling reactions.

13. (canceled)

14. The solution-phase process of claim 1, wherein the step of growing the oligonucleotide may be conducted in an organic solvent composition comprising at least one organic solvent.

15. The solution-phase process of claim 1, wherein the step of growing the oligonucleotide is conducted in neat acetonitrile or acetonitrile mixed with sulfolane (e.g., 4:1 v/v).

16. The solution-phase process of claim 1, wherein the growing oligonucleotide comprises:
at least one protected uracil nucleobase, wherein the protected uracil nucleobase comprises a protecting group; and/or
at least one protected thymine nucleobase, wherein the protected thymine nucleobase comprises a protecting group.

17. The solution-phase process of claim 16, wherein each protecting group is independently an acid-labile protecting group, a base labile protecting group, an ammonia labile protecting group, an oximate labile protecting group, an oxidatively labile protecting group, a hydrogenolytically labile protecting group or a transition metal catalysed cleavage protecting group.

18. The solution-phase process of claim 16, wherein each protecting group is independently selected from the group consisting of: 2,4,6-trimethylphenyl, 2-nitrophenyl, 2,4-dimethylphenyl, toluyl, 2-(4-nitrophenyl)ethyl, 2-(4-cyanophenyl)ethyl, allyl, benzoyl (Bz), 2,4-dimethylbenzoyl, tert-Bu benzoyl (tert-BuBz), acetyl (Ac), anisoyl (An), 4-chlorobenzoyl, diphenylcarbamoyl, butylthiocarbonyl, 2-nitrophenylsulfenyl, 2,4-dinitrophenylsulfenyl, 2-nitro-4-toluylsulfenyl, and triphenylmethylsulfenyl.

19. The solution-phase process of claim 16, wherein each protecting group is independently selected from the group consisting of benzoyl (Bz), 2,4-dimethylbenzoyl, tert-Bu benzoyl (tert-BuBz) and anisoyl (An).

20. The solution-phase process of claim 1, wherein the step of growing the oligonucleotide comprises a membrane filtration step after each sequential coupling reaction to isolate the growing oligonucleotide.

21. The solution-phase process of claim 1, wherein the one or more membrane filtration steps is performed using a crosslinked polybenzimidazole membrane or a polyetheretherketone membrane.

22. The solution-phase process of claim 1, wherein the process further comprises one or more deprotection steps.

23. (canceled)

24. The solution-phase process of claim 22, wherein the one or more deprotecting steps occurs during the step of growing the oligonucleotide and/or after the step of growing the oligonucleotide.

25. (canceled)
